(19) United States
(12) Patent Application Publication
(10) Pub. No.: US 2024/0254025 A1
(43) Pub. Date: Aug. 1, 2024

(54) SYSTEM AND METHOD FOR WATER ECOLOGICAL RESTORATION IN RURAL REGIONS

(71) Applicant: Nanjing Hydraulic Research Institute, Nanjing (CN)

(72) Inventors: Xiaojun WANG, Nanjing (CN); Jianyun ZHANG, Nanjing (CN); Feng CHEN, Nanjing (CN)

(21) Appl. No.: 18/634,567

(22) Filed: Apr. 12, 2024

(30) Foreign Application Priority Data
Apr. 18, 2023 (CN) 202310412370.5

(51) Int. Cl. C02F 3/32; C02F 1/00; C02F 3/00; C02F 103/00; G01N 33/18

(52) U.S. Cl. CPC C02F 3/322; C02F 1/004; C02F 3/006; C02F 3/327; G01N 33/18; C02F 2103/007; C02F 2201/009; C02F 2209/02

(57) ABSTRACT

A system for water ecological restoration in a rural region, including a water-collecting ditch assembly, a first biological treatment assembly, an ecological restoration assembly, an operation-regulating assembly and an energy supply assembly. The water-collecting ditch assembly is arranged in a yard. The first biological treatment assembly is internally communicated with the water-collecting ditch assembly. The ecological restoration assembly includes a restoration pool communicated with the water-collecting ditch assembly, a control valve provided on the restoration pool and a second biological treatment assembly communicated with the restoration pool. The restoration pool includes a main body and a spiral ditch component. The operation-regulating assembly is configured to regulate operating states of the water-collecting ditch assembly and the ecological restoration assembly. The energy supply assembly is electrically connected to the operation-regulating assembly. A method for water ecological restoration in a rural region is also provided.

### CROSS-REFERENCE TO RELATED APPLICATIONS

This application claims the benefit of priority from Chinese Patent Application No. 202310412370.5, filed on Apr. 18, 2023. The content of the aforementioned application, including any intervening amendments made thereto, is incorporated herein by reference in its entirety.

### TECHNICAL FIELD

This application relates to water ecological restoration and environmental protection, and more particularly to a system and method for water ecological restoration in rural regions.

### BACKGROUND

With the accelerated economic development in rural regions, environmental pollution problems, especially water environment pollution, have become increasingly prominent, and have become a major factor threatening the urban and rural living environment. At present, the discharge of domestic sewage in rural regions continues to increase, resulting in local problems such as shrinkage of water bodies, encroachment by reclamation, water pollution, and ecological degradation. Untreated domestic sewage causes serious pollution of various water sources and threatens the life and health of local residents. The rural regions have scattered population distribution and poor sewage treatment infrastructure. Some rural regions are only provided with pools to collect domestic sewage, which is treated by rainwater dilution and natural evaporation. Although sewage treatment plants have been built in some rural regions, the supporting facilities are still not in place, and the operation and maintenance costs are high. Therefore, expected benefits are not achieved. The existing technology lacks affordable, energy-efficient, and integrated water ecosystem restoration systems in rural regions.

### SUMMARY

An object of the disclosure is to provide a system and method for water ecological restoration in a rural region, so as to overcome the technical defect of the lack of water ecological restoration systems combined with rural regions in the prior art.

In order to achieve the above object, the following technical solutions are adopted.

In a first aspect, this application provides a system for water ecological restoration in a rural region, comprising a water-collecting ditch assembly, a first biological treatment assembly, an ecological restoration assembly, an operation-regulating assembly and an energy supply assembly; wherein the water-collecting ditch assembly is arranged in a yard; the first biological treatment assembly is internally communicated with the water-collecting ditch assembly; the ecological restoration assembly comprises a restoration pool communicated with the water-collecting ditch assembly, a control valve provided on the restoration pool, and a second biological treatment assembly communicated with the restoration pool; the restoration pool comprises a pool main body and a spiral ditch component; a first end of the spiral ditch component is communicated with the pool main body; and a second end of the spiral ditch component is communicated with the water-collecting ditch assembly through a water-converging ditch assembly; the operation-regulating assembly is configured to regulate an operating state of the water-collecting ditch assembly and the ecological restoration assembly; and the energy supply assembly is electrically connected to the operation-regulating assembly.

In some embodiments, the system further comprises an intermediate pool component; wherein a first end of the intermediate pool component is connected to the water-collecting ditch assembly; and a second end of the intermediate pool component is connected to the water-converging ditch assembly.

In some embodiments, the operation-regulating assembly comprises a plurality of water quality sensors, a plurality of flow velocity meters, a plurality of water pumps and a controlling component; the plurality of water quality sensors are each electrically connected to the energy supply assembly; and the plurality of water quality sensors are respectively arranged in the water-collecting ditch assembly, the restoration pool and the water-converging ditch assembly; the plurality of flow velocity meters are each electrically connected to the energy supply assembly; and the plurality of flow velocity meters are respectively arranged in the water-collecting ditch assembly, the restoration pool and the water-converging ditch assembly; the plurality of water pumps are each electrically connected to the energy supply assembly; and the plurality of water pumps are arranged in the water-collecting ditch assembly, the restoration pool and the water-converging ditch assembly; and the controlling component is electrically connected to the plurality of water quality sensors, the plurality of flow velocity meters, the plurality of water pumps and the energy supply assembly.

In some embodiments, the system further comprises a plurality of filtering assemblies; wherein the plurality of filtering assemblies are respectively arranged on the water-collecting ditch assembly, the restoration pool and the water-converging ditch assembly.

In some embodiments, each of the plurality of filtering assemblies comprises a housing, a driving component, a crushing structure and a travelling component; the housing is movably connected to the water-collecting ditch assembly, the restoration pool or the water-converging ditch assembly; and the housing is provided with an accommodating groove and a through hole communicated with the accommodating groove; the driving component is connected to the housing, and has a first use state and a second use state; the crushing structure is movably connected to the housing, and is configured to crush an object in the accommodating groove under driving of the driving component in the first use state; and the travelling component is connected to the housing, and is configured to drive the housing to move under driving of the driving component in the second use state.

In some embodiments, the system further comprises a temperature control assembly; wherein the temperature control assembly is configured to control a temperature of water bodies in the water-collecting ditch assembly, the restoration pool and the water-converging ditch assembly.

In some embodiments, the temperature control assembly comprises a lens structure, a heating wire and an insulating layer; the water-collecting ditch assembly, the restoration pool and the water-converging ditch assembly are each provided with the lens structure; the water-collecting ditch assembly, the restoration pool and the water-converging ditch assembly are each provided with the heating wire; and the heating wire is electrically connected to an external power supply; and the water-collecting ditch assembly, the restoration pool and the water-converging ditch assembly are each provided with the insulating layer.

In some embodiments, the energy supply assembly comprises a solar panel, a wind turbine power generation component and a power storage structure; the solar panel is movably connected to ground; the solar panel is in an arc shape; the solar panel has a first use state and a second use state; the solar panel is perpendicular to the ground in the first use state; and the solar panel is configured to partially cover the water-collecting ditch assembly, the restoration pool and the water-converging ditch assembly in the second use state; the wind turbine power generation component is connected to the solar panel; and the power storage structure is electrically connected to the solar panel, the wind turbine power generation component and the heating wire.

In a second aspect, this application provides a method for water ecological restoration in a rural region, the method comprising:
step (1) selecting a to-be-restored rural region, collecting and organizing hydrometeorology data, terrain elevation data and residential population data, constructing an ecological restoration layout for small and micro-water bodies based on a map of the to-be-restored rural region, and selecting native plants comprising emergent plants, floating plants, submerged plants and algae with strong adsorption capacity and at different spatial levels; wherein the hydrometeorology data comprises local rainfall data, regional water system, vegetation and sewage discharge; and the ecological restoration layout comprises layout of a water-collecting ditch, a water-converging ditch, a household intermediate pool and an ecological restoration pool;
step (2) building a water ecological restoration system based on conditions of the to-be-restored region, collecting and clarifying water quality index data for irrigation, greening, flushing and breeding, establishing a basic database and an internal-circulation regulation system, performing regular inspection on the water ecological restoration system and the internal-circulation regulation system to ensure that ditches are clear and devices are in normal operation, and regularly trimming the native plants, and collecting a nutrient in the native plants for resource utilization; and
step (3) introducing weather forecast data into the local rainfall data, and pumping substandard sewage into a spiral ditch for internal circulation treatment or into the ecological restoration pool for treatment followed by utilization or discharge until a water quality reaches a utilization or discharge standard.

Compared with the prior art, this disclosure has the following beneficial effects. The system and method of the present disclosure adopt a cooperation of the water-collecting ditch assembly, the first biological treatment assembly, the ecological restoration assembly and the energy supply assembly to realize the interception and utilization of scattered domestic sewage in the to-be-restored region and reduce non-point source pollution. The first biological treatment assembly and the second biological treatment assembly adopt comprehensive biological measures such as the native plants and algae to treat the domestic sewage, resulting in a reduction in treatment consumption, making secondary pollution less likely to occur, increasing local biodiversity, and effectively improving local microclimate. The connection of the small and micro-water bodies in the rural region facilitates the avoidance of black and smelly water bodies. Treated and standardized water is configured for local ecological greening, flushing and breeding. An efficient use of internal water is achieved through circulation, thereby saving water resources. An efficient use of existing local resources depending on rules of regional topography, weather, plant growth, etc. can effectively improve the rural water environment.

### BRIEF DESCRIPTION OF THE DRAWINGS

In order to explain the technical solutions in the embodiments of the present disclosure more clearly, the accompanying drawings required in the description of the embodiments or prior art will be briefly introduced below. Obviously, presented in the drawings are merely some embodiments of the disclosure. For those of ordinary skill in the art, other drawings can be obtained based on the structures illustrated herein without making creative efforts.

FIG. 1 is a flow chart illustrating operation of a system for water ecological restoration in a rural region in accordance with an embodiment of the present disclosure;

FIG. 2 is a structural diagram of the system in accordance with an embodiment of the present disclosure;

FIG. 3 is a sectional view of a restoration pool of the system in accordance with an embodiment of the present disclosure;

FIG. 4 is a schematic diagram of a first connection state between a water-collecting ditch assembly and a first biological treatment assembly of the system in accordance with an embodiment of the present disclosure;

FIG. 5 is a schematic diagram of a second connection state between the water-collecting ditch assembly and the first biological treatment assembly in accordance with an embodiment of the present disclosure;

FIG. 6 is a front view of the water-collecting ditch assembly and the first biological treatment assembly in the second connection state depicted in FIG. 5; and

FIG. 7 is a structural diagram of a filtering assembly of the system in accordance with an embodiment of the present disclosure.

In the drawings: 1. water-collecting ditch assembly; 2. first biological treatment assembly; 3. restoration pool; 31. pool main body; 32. spiral ditch component; 4. control valve; 5. second biological treatment assembly; 6. water-converging ditch assembly; 7. operation-regulating assembly; 71. water quality sensor; 72. flow velocity meter; 8. energy supply assembly; 81. solar panel; 82. power storage structure; 9. filtering assembly; 91. housing; 92. driving component; 93. crushing structure; 94. travelling component; 10. intermediate pool component; and 100. temperature control assembly.

### DETAILED DESCRIPTION OF EMBODIMENTS

In order to make the to-be-solved technical problems, technical solutions, beneficial effects of the present disclosure clearer, this application will be described in further detail below with reference to the accompanying drawings and embodiments. It should be understood that the specific embodiments described herein are merely intended to explain the present disclosure, and are not intended to limit the present disclosure.

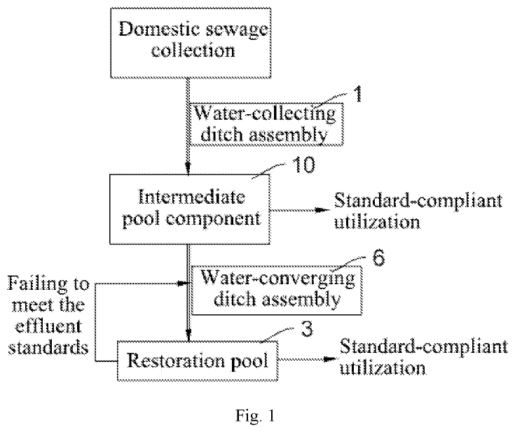

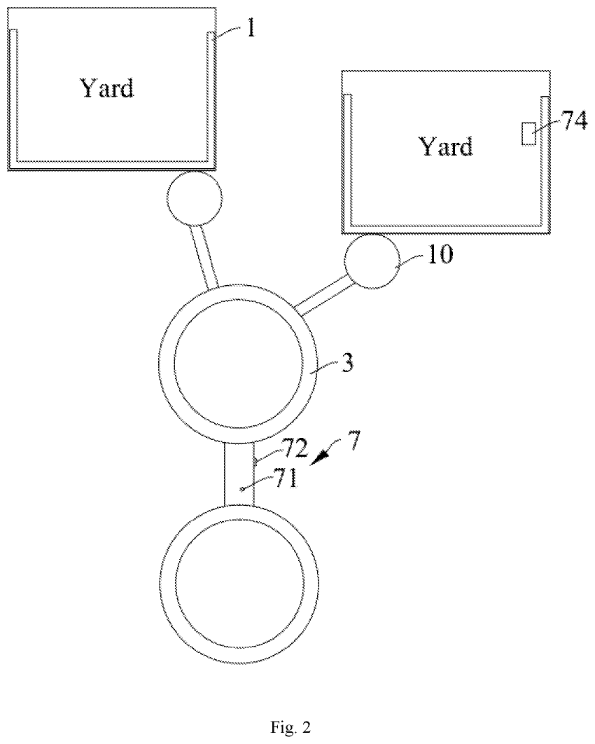

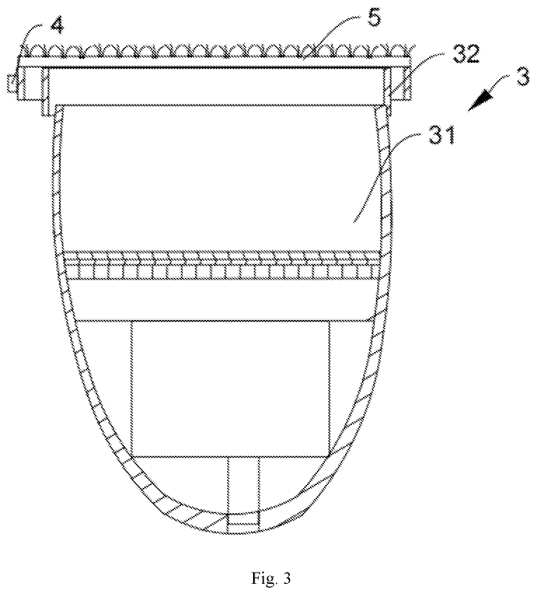

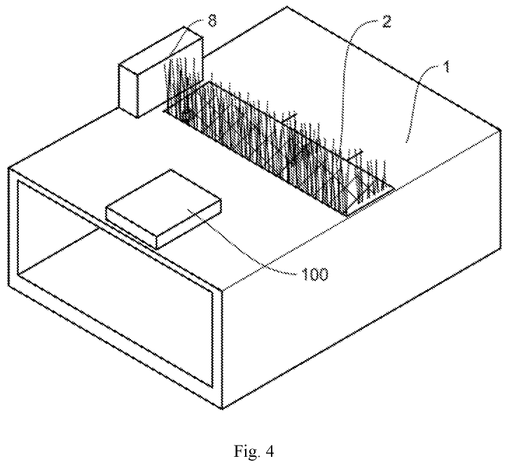

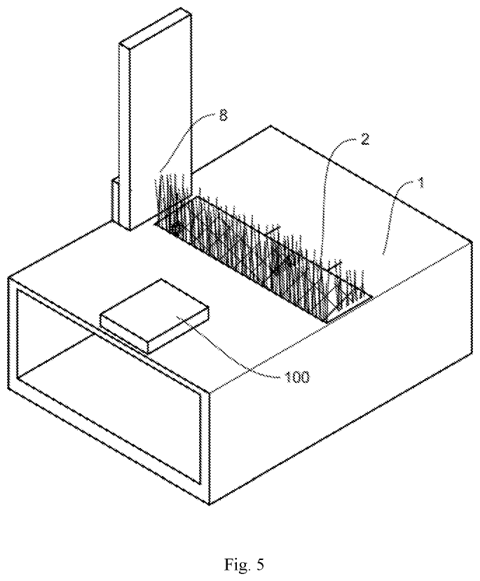

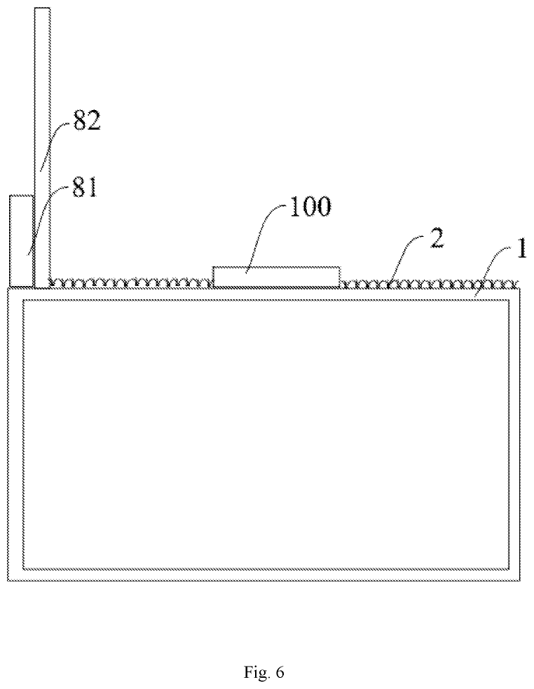

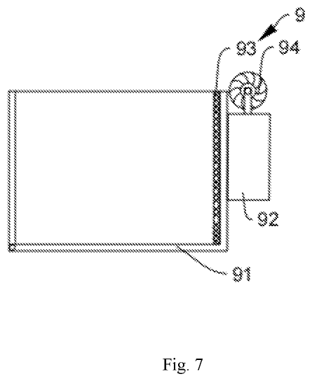

Referring to FIGS. 1-7, a system for water ecological restoration in a rural region is provided, which includes a water-collecting ditch assembly 1, a first biological treatment assembly 2, an ecological restoration assembly, an operation-regulating assembly 7 and an energy supply assembly 8. The water-collecting ditch assembly 1 is arranged in a yard. The first biological treatment assembly 2 is internally communicated with the water-collecting ditch assembly 1. The ecological restoration assembly includes a restoration pool 3 communicated with the water-collecting ditch assembly, a control valve 4 provided on the restoration pool 3, and a second biological treatment assembly 5 communicated with the restoration pool 3. The restoration pool 3 includes a pool main body 31 and a spiral ditch component 32. A first end of the spiral ditch component 32 is communicated with the pool main body 31. A second end of the spiral ditch component 32 is communicated with the water-collecting ditch assembly 1 through a water-converging ditch assembly 6. The operation-regulating assembly 7 is configured to regulate an operating state of each of the water-collecting ditch assembly 1 and the ecological restoration assembly. The energy supply assembly 8 is electrically connected to the operation-regulating assembly 7.

The water-collecting ditch assembly 1 and the first biological treatment assembly 2 together constitute a household sewage collection and treatment assembly, which is arranged with the rural household as basic unit. The water-collecting ditch assembly 1 is arranged around the yard. The first biological treatment assembly 2 is provided above and on both sides of the water-collecting ditch assembly 1. The first biological treatment assembly 2 is dominated by native plants, including emergent plants, floating plants, submerged plants and algae.

The water-collecting ditch assembly 1 includes a water-collecting ditch main body. A water passing capacity Q_{iW} of a cross-section of the water-collecting ditch main body is expressed as: Q_{iW}=max(Q_{iR}, Q_{iS}), where Q_{iR} is rainfall amount of a preset range, Q_{iS} is amount of domestic sewage collected within the preset range, and i represents the first, second, third, . . . , or n-th household. Generally, the rainy areas are dominated by Q_{iR}, and the dry areas with less rain are dominated by Q_{iS}.

In this embodiment, the water-collecting ditch main body is U-shaped. The water-collecting ditch assembly 1 further includes a top cover for covering the water-collecting ditch main body. The top cover can be arranged on the water-collecting ditch main body according to actual needs.

The pool main body 31 of the restoration pool is laid out according to a topography and adopting an original low-lying land or a pond within a regional scope. The spiral ditch component 32 is provided on an upper portion of the pool main body 31. The arrangement of the spiral ditch component 32 facilitates extending a sewage treatment time. The control valve 4 is provided at each 10-50 m interval on the spiral ditch component 32. An upper side and both side face of the restoration pool 3 are each provided with the second biological treatment assembly 5. The second biological treatment assembly 5 is dominated by native plants, including emergent plants, floating plants, submerged plants and algae. The second biological treatment assembly 5 contains native plants and algae with strong adsorption capability.

The spiral ditch component 32 includes a spiral ditch main body. A bottom elevation of the spiral ditch main body gradually decreases from outside to inside, which facilitates extending a sewage treatment time. A baffle is provided on an inner side of the spiral ditch main body, and is made of an anti-corrosion and penetration material with a certain strength, such as a steel plate and an engineering plastic.

The spiral ditch main body can be arranged in 2-4 rows according to a size of the pool main body 31 of the restoration pool. The spiral ditch is planted with plants to increase a restoration contact surface and extend a restoration time.

The operation-regulating assembly 7 is configured to achieve recycling recovery and internal efficient utilization of sewage.

Compared with the prior art, the system provided in this application adopts the cooperation of the water-collecting ditch assembly 1, the first biological treatment assembly 2, the ecological restoration assembly and the energy supply assembly 8 to realize the interception and utilization of scattered domestic sewage in the rural region and reduce non-point source pollution. The first biological treatment assembly 2 and the second biological treatment assembly 5 adopt comprehensive biological measures such as native plants and algae to treat the domestic sewage, resulting in a reduction in treatment consumption, making secondary pollution less likely to occur, increasing local biodiversity, and effectively improving local microclimate. The connection of small and micro-water bodies in the rural region facilitates the avoidance of black and smelly water bodies. Treated and standardized water is configured for local ecological greening, flushing and breeding. An efficient use of internal water is achieved through circulation, thereby saving water resources. An efficient use of existing local resources depending on rules of regional topography, weather, plant growth, etc. can effectively improve the rural water environment.

In this embodiment, the system further includes an intermediate pool component 10. A first end of the intermediate pool component 10 is connected to the water-collecting ditch assembly 1. A second end of the intermediate pool component 10 is connected to the water-converging ditch assembly 6. The restoration pool 3 is connected to the intermediate pool component through the water-converging ditch assembly 6. A water passing capacity of a water-converging ditch main body of the water-converging ditch assembly 6 is expressed as: Q_{iC}=max (Q_{iE},Q_{iT}), where Q_{iE} is water volume of the restoration pool 3, Q_{iT} is water volume of the intermediate pool component 10.

A total sewage treatment capacity Qᵣₛ of the system is calculated as:

$${Q}_{TS} = {\sum }_{i = s}^{x} {Q}_{iE} ,$$

which is the sum of water volumes of all restoration pools.

The intermediate pool component 10 is arranged according to terrain and other conditions, satisfying Q_{iT}≤Q_{iW}. Plants in and around the intermediate pool component 10 are treated in the same way as the water-collecting ditch assembly 1.

In an embodiment of the disclosure, as shown in FIGS. 1-7, the operation-regulating assembly 7 includes a plurality of water quality sensors 71, a plurality of flow velocity meters 72, a plurality of water pumps and a controlling component. The plurality of water quality sensors 71 are each electrically connected to the energy supply assembly 8. The plurality of water quality sensors 71 are respectively arranged in the water-collecting ditch assembly 1, the restoration pool 3 and the water-converging ditch assembly 6. The plurality of flow velocity meters 72 are each electrically connected to the energy supply assembly 8. The plurality of flow velocity meters 72 are respectively arranged in the water-collecting ditch assembly 1, the restoration pool 3 and the water-converging ditch assembly 6. The plurality of water pumps are electrically connected to the energy supply assembly 8. The plurality of water pumps are respectively arranged in the water-collecting ditch assembly 1, the restoration pool 3 and the water-converging ditch assembly 6. The controlling component is electrically connected to the plurality of water quality sensors 71, the plurality of flow velocity meters 72, the plurality of water pumps and the energy supply assembly 8.

An inlet and an outlet of each of the restoration pool 3 and the intermediate pool component 10 are each provided with an inlet and outlet flow rate detection device, a water quality monitoring device, a filter, a barrier and a pumping and drainage device. The plurality of water quality sensors 71 are configured for real-time monitoring of water pollutants at a fixed point. The plurality of flow velocity meters 72 are configured to measure a flow velocity. The barrier is configured to control an amount of water entering and exiting. The pool main body 31 and the intermediate pool component 10 are each provided with a water pump configured for water transfer and drainage between the restoration pool 3 and the water-converging ditch assembly 6, the intermediate pool component 10 and the water-collecting ditch assembly 1, so as to achieve efficient utilization.

A total domestic water demand Ry for the rural region (excluding drinking water) is calculated through the following equation: Rₜₒₜₐₗ=R_{ecology}+R_{flush}+R_{breed}, where R_{ecology} is the ecological water demand within a range of 1-5 m around the ditches and ponds, R_{flush} is water demand for flushing, and R_{breed} refers to water demand for breeding. R_{ecology} is calculated as R_{ecology}=S□h□(θ_{hold}−θ_{wither}), where R_{ecology} is the ecological water demand within a range of 1-5 m around the ditches and ponds, S is an area of surrounding trees, shrubs and grass, h is the depth of plant root system ranging from about 10 cm to 80 cm, θ_{hold} is saturated water holding capacity of soil, and θ_{wither} is wither moisture content of the soil.

On the basis that the water quality meets the standard, a priority of water supply satisfies R_{ecology}≥R_{breed}≥R_{flush}.

In the system for water ecological restoration in the rural region, a flow of water bodies is maintained through terrain elevation design and water pump drainage.

In an embodiment, as shown in FIG. 1-7, the system further includes a plurality of filtering assemblies 9. The plurality of filtering assemblies 9 are respectively arranged on the water-collecting ditch assembly 1, the restoration pool 3 and the water-converging ditch assembly 6. The plurality of filtering assemblies 9 are configured to filter and purify water flowing into the system to reduce a risk of being blocked.

In this embodiment, each of the plurality of filtering assemblies 9 includes a housing 91, a driving component 92, a crushing structure 93 and a travelling component 94. The housing 91 is movably connected to the water-collecting ditch assembly 1 or the restoration pool 3 or the water-converging ditch assembly 6. The housing 91 is provided with an accommodating groove and a connecting hole connected to the accommodating groove. The driving component 92 is connected to the housing 91, and has a first use state and a second use state. The crushing structure 93 is movably connected to the housing 91, and is configured to crush an object in the accommodating groove under driving of the driving component 92 in the first use state. The travelling component 94 is connected to the housing 91, and is configured to drive the housing 91 to move under driving of the driving component 92 in the second use state.

In this embodiment, the housing 91 includes a frame body and a movable plate movably connected to the frame body. An upper end surface and any one of side surfaces of the frame body are each an open-end surface. The movable plate has a first use state and a second use state. The movable plate in the first use state is in sealed connection with a side surface, and the side surface is the open-end surface. The movable plate in the second use state is connected to the upper end surface. The frame body is provided with a groove component for limiting the movable plate.

The driving component 92 includes a motor, a screw rod and a movable block. The motor is fixedly connected to the housing 91. The screw rod is arranged in the accommodating groove. One end of the screw rod is connected to a driving end of the motor. The movable block is threadedly connected to the screw rod. The screw rod is connected to the driving end of the motor through a connecting component. The crushing structure 93 includes a frame adapted to the accommodating groove, a crushing net fixed on the frame and a storage box movably connected to a main body of the frame. The storage box is configured to store debris after being crushed by the crushing net. The frame is connected to the movable block.

As an example, the filtering assembly 9 is mounted in the water-collecting ditch assembly 1 with the movable plate in the first use state. The upper end surface of the frame body is the open-end surface. Sewage containing debris is poured from the open-end surface and directly into the water-collecting ditch assembly 1 through the connecting hole of the housing 91. When the filtering assembly 9 contains an impurity (such as a fallen leaf, a peel, a plastic bag, etc.), the driving component 92 can be started to drive the crushing structure 93 to move. The peel is crushed by the crushing structure 93. Then, the frame body is flushed. The peel is brought into the storage box under the action of water flow. The plastic bag cannot be broken by the crushing structure 93. The broken peel is taken out from the frame body and processed.

The travelling component 94 includes a traveling wheel rotatably connected to the housing 91.

The connecting component has a first use state and a second use state. The connecting component in the first use state is connected to the screw rod. The connecting component in the second use state is connected to the traveling wheel.

The connecting component includes a first connecting rod connected to the motor and a second connecting rod movably connected to the first connecting rod. A meshing wheel is provided at one end of the second connecting rod away from the first connecting rod. The meshing wheel is configured to mesh with the screw rod in the first use state of the connecting component. The meshing wheel is configured to mesh with the traveling wheel in the second use state of the connecting component. The first connecting rod is provided with a first connection position and a second connection position. When the second connecting rod is connected to the first connection position, the connecting component is in the first use state. When the second connecting rod is connected to the second connecting position, the connecting component is in the second use state.

In an embodiment, as shown in FIGS. 1-7, the system further includes a temperature control assembly 100. The temperature control assembly 100 is configured to control a temperature of water bodies in the water-collecting ditch assembly 1, the restoration pool 3 and the water-converging ditch assembly 6. The temperature control assembly 100 enables the system to be used normally even in a cold area.

In this embodiment, the temperature control assembly 100 includes a lens structure, a heating wire and an insulating layer. The water-collecting ditch assembly 1, the restoration pool 3 and the water-converging ditch assembly 6 are each provided with the lens structure. The water-collecting ditch assembly 1, the restoration pool 3 and the water-converging ditch assembly 6 are each provided with the heating wire. The heating wire is electrically connected to an external power supply. The water-collecting ditch assembly 1, the restoration pool 3 and the water-converging ditch assembly 6 are each provided with the insulating layer.

The energy supply of the system adopts wind energy, solar energy and battery. The energy supply assembly 8 includes a solar panel 81, a wind turbine power generation component and a power storage structure 82. The solar panel 81 is movably connected to ground. The solar panel 81 is in an arc shape. The solar panel 81 has a first use state and a second use state. The solar panel 81 is perpendicular to the ground in the first use state. The solar panel 81 is configured to partially cover the water-collecting ditch assembly 1, the restoration pool 3 and the water-converging ditch assembly 6 in the second use state. The wind turbine power generation component is connected to the solar panel 81. The power storage structure 82 is electrically connected to the solar panel 81, the wind turbine power generation component and the heating wire.

A method for water ecological restoration in a rural region is provided, which includes the following steps.

Step (1) A to-be-restored rural region is selected. Hydrometeorology data, terrain elevation data and residential population data are collected and organized. The hydrometeorology data mainly includes local rainfall data, regional water systems, vegetation and sewage discharge. An ecological restoration layout for small and micro-water bodies is constructed based on a map of the to-be-restored rural region. The ecological restoration layout includes layout of a water-collecting ditch, a water-converging ditch, a household intermediate pool and an ecological restoration pool. Native plants including emergent plants, floating plants, submerged plants and algae with strong adsorption capacity and at different spatial levels are selected.

Step (2) A water ecological restoration system is built based on conditions of the to-be-restored rural region. Water quality index data for irrigation, greening, flushing and breeding is collected and clarified. A basic database and an internal-circulation regulation system are established. A regular inspection is performed on the water ecological restoration system and the internal-circulation regulation system to ensure that ditches are clear, and devices are in normal operation. The native plants are regularly trimmed. A nutrient in the native plants is collected for resource utilization.

Step (3) The weather forecast data collected in the past 5 to 10 years is introduced into the local rainfall data. Substandard sewage is pumped into a spiral ditch for internal circulation treatment or into the ecological restoration pool for treatment followed by utilization or discharge until a water quality reaches a utilization or discharge standard.

Described above are merely some preferred embodiments of the present disclosure, and are not intended to limit the present disclosure. It should be understood that various modifications, replacements and improvements made by those skilled in the art without departing from the spirit of the disclosure shall fall within the scope of the present disclosure defined by the appended claims.

What is claimed is:

1. A system for water ecological restoration in rural region, comprising:
a water-collecting ditch assembly;
a first biological treatment assembly;
an ecological restoration assembly;
an operation-regulating assembly; and
an energy supply assembly;
wherein the water-collecting ditch assembly is arranged in a yard;
the first biological treatment assembly is internally communicated with the water-collecting ditch assembly;
the ecological restoration assembly comprises a restoration pool communicated with the water-collecting ditch assembly, a control valve provided on the restoration pool, and a second biological treatment assembly communicated with the restoration pool; the restoration pool comprises a pool main body and a spiral ditch component; a first end of the spiral ditch component is communicated with the pool main body; and a second end of the spiral ditch component is communicated with the water-collecting ditch assembly through a water-converging ditch assembly;
the operation-regulating assembly is configured to regulate an operating state of the water-collecting ditch assembly and the ecological restoration assembly; and
the energy supply assembly is electrically connected to the operation-regulating assembly.

2. The system of claim 1, further comprising:
an intermediate pool component;
wherein a first end of the intermediate pool component is connected to the water-collecting ditch assembly; and a second end of the intermediate pool component is connected to the water-converging ditch assembly.

3. The system of claim 1, wherein the operation-regulating assembly comprises a plurality of water quality sensors, a plurality of flow velocity meters, a plurality of water pumps and a controlling component;
the plurality of water quality sensors are each electrically connected to the energy supply assembly; and the plurality of water quality sensors are respectively arranged in the water-collecting ditch assembly, the restoration pool and the water-converging ditch assembly;
the plurality of flow velocity meters are each electrically connected to the energy supply assembly; and the plurality of flow velocity meters are respectively arranged in the water-collecting ditch assembly, the restoration pool and the water-converging ditch assembly;
the plurality of water pumps are each electrically connected to the energy supply assembly; and the plurality of water pumps are arranged in the water-collecting ditch assembly, the restoration pool and the water-converging ditch assembly; and
the controlling component is electrically connected to the plurality of water quality sensors, the plurality of flow velocity meters, the plurality of water pumps and the energy supply assembly.

4. The system of claim 1, further comprising:
a plurality of filtering assemblies;
wherein the plurality of filtering assemblies are respectively arranged on the water-collecting ditch assembly, the restoration pool and the water-converging ditch assembly.

5. The system of claim 4, wherein each of the plurality of filtering assemblies comprises a housing, a driving component, a crushing structure and a travelling component;
the housing is movably connected to the water-collecting ditch assembly, the restoration pool or the water-converging ditch assembly; and the housing is provided with an accommodating groove and a through hole communicated with the accommodating groove;
the driving component is connected to the housing, and has a first use state and a second use state;
the crushing structure is movably connected to the housing, and is configured to crush an object in the accommodating groove under driving of the driving component in the first use state; and
the travelling component is connected to the housing, and is configured to drive the housing to move under driving of the driving component in the second use state.

6. The system of claim 1, further comprising:
a temperature control assembly;
wherein the temperature control assembly is configured to control a temperature of water bodies in the water-collecting ditch assembly, the restoration pool and the water-converging ditch assembly.

7. The system of claim 6, wherein the temperature control assembly comprises a lens structure, a heating wire and an insulating layer;
the water-collecting ditch assembly, the restoration pool and the water-converging ditch assembly are each provided with the lens structure;
the water-collecting ditch assembly, the restoration pool and the water-converging ditch assembly are each provided with the heating wire; and the heating wire is electrically connected to an external power supply; and
the water-collecting ditch assembly, the restoration pool and the water-converging ditch assembly are each provided with the insulating layer.

8. The system of claim 7, wherein the energy supply assembly comprises a solar panel, a wind turbine power generation component and a power storage structure;
the solar panel is movably connected to ground; the solar panel is in an arc shape; the solar panel has a first use state and a second use state; the solar panel is perpendicular to the ground in the first use state; and the solar panel is configured to partially cover the water-collecting ditch assembly, the restoration pool and the water-converging ditch assembly in the second use state;
the wind turbine power generation component is connected to the solar panel; and
the power storage structure is electrically connected to the solar panel, the wind turbine power generation component and the heating wire.

9. A method for water ecological restoration in rural region, comprising:
step (1) selecting a to-be-restored rural region, collecting and organizing data on hydrometeorology, terrain elevation, residential population, regional water system, vegetation and sewage discharge, constructing an ecological restoration layout for small and micro-water bodies such as rural ponds and ditches based on a map of the to-be-restored rural region, and selecting native plants comprising emergent plants, floating plants, submerged plants and algae with strong adsorption capacity and at different spatial levels; wherein the hydrometeorology data comprises local rainfall data; and the ecological restoration layout comprises layout of a water-collecting ditch, a water-converging ditch, a household intermediate pool and an ecological restoration pool;
step (2) building a water ecological restoration system based on conditions of the to-be-restored rural region, collecting and clarifying water quality index data for irrigation, greening, flushing and breeding, establishing a basic database and an internal-circulation regulation system, performing regular inspection on the water ecological restoration system and the internal-circulation regulation system to ensure that ditches are clear and devices are in normal operation, and regularly trimming the native plants, and collecting a nutrient in the native plants for resource utilization; and
step (3) introducing weather forecast data into the local rainfall data, and pumping substandard sewage into a spiral ditch for internal circulation treatment or into the ecological restoration pool for treatment followed by utilization or discharge until a water quality reaches a utilization or discharge standard.
